(19) United States
(12) Patent Application Publication
(10) Pub. No.: US 2023/0113763 A1
(43) Pub. Date: Apr. 13, 2023

(54) Eye mask with integrally formed heating structure and its manufacturing process

(71) Applicant: Dongguan Jiwo Electronic Technology Co. Ltd, Dongguan (CN)

(72) Inventor: Yingxu Zhang, Meishan (CN)

(73) Assignee: Dongguan Jiwo Electronic Technology Co. Ltd, Dongguan, GD (CN)

(21) Appl. No.: 18/079,914

(22) Filed: Dec. 13, 2022

(51) Int. Cl. A61F 7/00

(52) U.S. Cl. CPC A61F 7/007; A61F 2007/0078; A61F 2007/0004

(57) ABSTRACT

The invention provides an eye mask with an integrally formed heating structure and a manufacturing process thereof, belonging to the field of nursing appliances. It includes an eye mask main body manufactured by an integral molding process, a detachable power supply controller, and a plug-in power supply mechanism. Wherein, the main body of the eye mask is sewn from non-woven fabric, sponge, and carbon fiber heating body. The present invention can quickly sew an eye mask with an integrated structure by using the cut non-woven fabric, sponge, and carbon fiber heating body so that the processing method of the eye mask is simpler and the manufacturing cost is reduced, and the eye mask using the carbon fiber heating body can realize constant temperature heating.

### TECHNICAL FIELD

The invention relates to the field of nursing appliances, in particular, it relates to an eye mask with an integrally formed heating structure and a manufacturing process thereof.

### BACKGROUND

As far as the electric heating eye mask is concerned, it achieves heating and heating through the heating element, which has high safety and good reusability and has been recognized by the public.

But existing electric heating type heating eye masks all is to adopt the structure of a built-in battery usually. In the processing eye masks, the manufacturing process is relatively complicated and the manufacturing cost is high, and heating eye masks with built-in batteries are also inconvenient in daily cleaning and maintenance. Therefore, there is a good market prospect in providing an eye mask with an integrally formed heating structure that is convenient for processing and daily use.

### SUMMARY

Embodiments of the present invention provide an eye mask with an integrated heating structure and its manufacturing process, aiming to solve the problems of complex structure and certain inconveniences in the use of the existing eye mask.

An embodiment of the present invention provides an eye mask with an integrated heating structure, including an eye mask main body manufactured by an integral molding process, a detachable power supply controller, and a plug-in power supply mechanism.

Wherein, the main body of the eye mask of the present invention is sewn from non-woven fabric, sponge, and carbon fiber heating body;

The power supply controller of the present invention is composed of an outer shell, a display panel, a PCB control board, a storage battery, and an elastic locking mechanism, wherein the elastic locking mechanism is buckled with the socket provided outside the eye mask host;

The plug-in power supply mechanism of the present invention is composed of a female head installed inside the socket and a male head installed inside the outer casing; after the male head and the female head are plugged together, they provide electric energy for the carbon fiber heating body and make the main body of the eye mask generate heat.

In this embodiment, the non-woven fabric, sponge, and carbon fiber heating body are cut into the shape of an eye mask, wherein both the non-woven fabric and the sponge have at least two layers; the carbon fiber heating body is placed between two layers of sponge; two layers of non-woven fabric The exterior of the two layers of foam is sewn into a one-piece eye mask.

In one embodiment of the utility model, the present invention cuts the non-woven fabric, the sponge, and the carbon fiber heating body into the shape of an eye mask, and then uses the sewing machine to sew it into the main body of the eye mask.

In this embodiment, the socket includes a base fixedly installed on the outer layer of non-woven fabric; the top of the base is fixedly connected with a boss; the edge of the boss is provided with an annular groove; a groove is reserved in the middle of the boss, and the female head is fixedly installed in the groove Inside.

In one embodiment of the utility model, the utility model realizes the installation of the socket and the main body of the eye mask by fixing the female head on the inner side of the card slot and then fixing the base on the outer layer of non-woven fabric.

In this embodiment, the display panel is installed on the front of the casing, and the PCB control board, battery, and elastic locking mechanism are all installed inside the casing; the power switch and charging port are also installed on both sides of the casing.

In one embodiment of the utility model, the PCB control board, the storage battery and the elastic locking mechanism are installed inside the outer casing, so that the power supply controller can be miniaturized and integrated.

In this embodiment, the elastic locking mechanism includes a pressing block plugged into the top of the outer casing; one end of the pressing block is connected with a sliding piece placed inside the outer casing; the bottom end of the sliding piece is fixedly connected with two root guide rod;The outside of the guide rod is covered with a return spring fixedly connected to the inside of the outer shell; a lock hole is opened in the middle of the slide piece; a section of the edge of the lock hole is engaged with the annular groove;a guide chute is provided inside the outer casing at a position directly opposite to the slide; the back of the slide is slidably connected to the guide chute.

In one embodiment of the utility model, by pressing the pressing block, the lock hole can be unlocked with the socket, and the power supply controller can be quickly disassembled.

The manufacturing process of an eye mask with an integrally formed heating structure, which is used to manufacture the eye mask described in any one of the above, specifically includes the following steps:

S1, the present invention selects two layers of non-woven fabrics, two layers of sponges, and a carbon fiber heating body for cutting, and cuts into the shape of an eye mask;

S2, the present invention sews the cut non-woven fabric, sponge, and carbon fiber heating body to form an integrated eye mask with a heating function;

S3, the present invention sews Velcro at the two ends of the inner non-woven fabric;

S4, the present invention installs a socket with a female head on the outer non-woven fabric;

S5, the present invention buckles the elastic locking mechanism of the power supply controller with the socket to complete the detachable installation of the power supply controller.

Compared with the prior art, the beneficial effect of the present invention is:

The present invention can quickly sew an eye mask with an integrated structure by using the cut non-woven fabric, sponge, and carbon fiber heating body so that the processing method of the eye mask is simpler and the manufacturing cost is reduced, and the eye mask using the carbon fiber heating body can realize constant temperature heating. This can promote blood circulation around the eyes, improve eye fatigue, and relax eye muscles;The grooves on the inside of the eye mask's main body can be used with ice packs to apply ice to the eyes, relieve eye swelling and swelling, and better release eye pressure. At the same time, the blindfold is fastened to the elastic locking mechanism of the power supply controller through the provided socket, so that the power supply controller can be easily disassembled while realizing the power supply control. After the power supply controller is disassembled, the eye mask can be cleaned and maintained more conveniently, and the practicability of the eye mask can be improved.

### BRIEF DESCRIPTION OF THE DRAWINGS

To illustrate the technical solutions of the embodiments of the present invention more clearly, the following will briefly introduce the drawings that are used in the embodiments. It should be understood that the following drawings only illustrate certain embodiments of the invention and therefore should not be considered as limiting the scope. For those skilled in the art, other related drawings can also be obtained based on these drawings without creative effort.

FIG. 1 is a schematic structural view of an eye mask with an integrally formed heating structure provided by an embodiment of the present invention;

FIG. 2 is a schematic diagram of a partial structure of an eye mask with an integrally formed heating structure provided by an embodiment of the present invention;

FIG. 3 is a cross-sectional view of the main body of the eye mask provided by the embodiment of the present invention;

FIG. 4 is a schematic structural diagram of a power supply controller provided by an embodiment of the present invention;

FIG. 5 is one of the structural schematic diagrams of the power supply controller provided by the embodiment of the present invention;

FIG. 6 is a schematic diagram of the internal structure of the power supply controller provided by the embodiment of the present invention;

FIG. 7 is a schematic structural diagram of the elastic locking mechanism provided by the embodiment of the present invention;

FIG. 8 is a schematic diagram of the structure of the socket provided by the embodiment of the present invention.

Icons: 10. An eye mask with integrally formed heating structure; 100. Eye mask host; 110. Non-woven fabric; 111. Velcro; 120. Sponge; 130. Carbon fiber heating body;140. Socket; 141. Base; 142. Convex seat; 143. Annular groove; 144. Slotting; 150. Groove; 200. Power supply controller;210. Outer casing; 220. Display panel; 230. PCB control board; 240. Battery; 250. Elastic locking mechanism; 251. Pressing block; 252. Slider;253. Guide rod; 254. Return spring; 255. Keyhole; 260. Power switch; 270. Charging port; 280. Guide chute; 300. Plug-in power supply mechanism; 310. Female head;

### DETAILED DESCRIPTION

To make the purpose, technical solutions, and advantages of the embodiments of the present invention clearer, the technical solutions in the embodiments of the present invention will be clearly and completely described below in conjunction with the drawings in the embodiments of the present invention. The described embodiments are some but not all of the embodiments of the present invention. Based on the implementation manners in the present invention, all other implementation manners obtained by persons of ordinary skill in the art without creative efforts fall within the protection scope of the present invention.

Accordingly, the following detailed description of the embodiments of the invention provided in the accompanying drawings is not intended to limit the scope of the claimed invention, but merely represents selected embodiments of the invention. Based on the implementation manners in the present invention, all other implementation manners obtained by persons of ordinary skill in the art without creative efforts fall within the protection scope of the present invention.

It should be noted that like numerals and letters denote similar items in the following Figures, therefore, once an item is defined in one Figure, it does not require further definition and explanation in subsequent Figures.

In describing the present invention, it should be understood that the terms “center”, “longitudinal”, “transverse”, “length”, “width”, “thickness”, “upper”, “lower”, “front”, “Orientation indicated by the rear, left, right, vertical, horizontal, top, bottom, inside, outside, clockwise, counterclockwise, etc. The positional relationship is based on the orientation or positional relationship shown in the drawings. This is only for the convenience of describing the present invention and simplifying the description, but does not indicate or imply that the referred device or element must have a specific orientation, be constructed and operated in a specific orientation, and thus should not be construed as limiting the present invention.

In addition, the terms “first” and “second” are used for descriptive purposes only, and cannot be interpreted as indicating or implying relative importance or implicitly specifying the quantity of indicated technical features. Thus, a feature defined as “first” and “second” may explicitly or implicitly include one or more of these features. In the description of the present invention, “plurality” means two or more, unless otherwise specifically defined.

In the present invention, terms such as “mounting”, “connecting”, “connecting” and “fixing” should be interpreted in a broad sense unless otherwise clearly specified and limited. For example, it can be a fixed connection, a detachable connection, or an integrated one; it can be directly connected, or indirectly connected through an intermediary; it can be the internal communication of two elements or the interaction relationship between two elements. Those of ordinary skill in the art can understand the specific meanings of the above terms in the present invention according to specific situations.

In the present invention, unless otherwise clearly specified and limited, a first feature is “on” or “under” a second feature may include direct contact between the first and second features, and may also include the first and second features Not in direct contact but through another characteristic contact between them. Moreover, “above”, “above” and “above” the first feature on the second feature include that the first feature is directly above and obliquely above the second feature, or simply means that the first feature is higher in level than the second feature. “Below”, “under” and “under” the first feature to the second feature include that the first feature is directly below and obliquely below the second feature, or simply means that the first feature is less horizontal than the second feature.

### APPLICABILITY

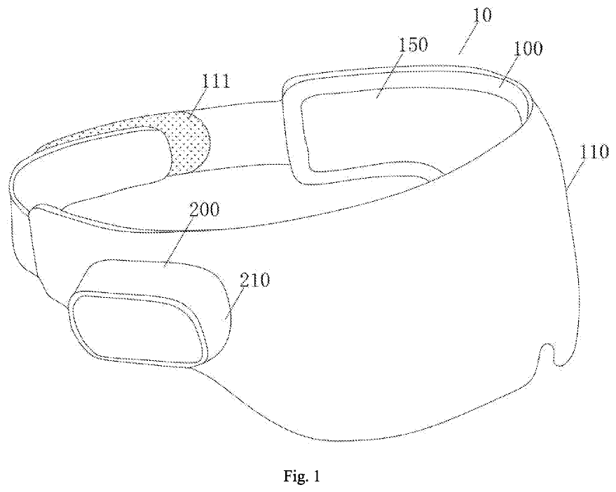

Please refer to FIG. 1: an eye mask 10 with an integrated heating structure, including an eye mask main body 100 manufactured by an integral molding process, a detachable power supply controller 200, and a plug-in power supply mechanism 300.

Refer to FIGS. 1-3 for details: the eye mask host 100 is sewn from a non-woven fabric 110, a sponge 120, and a carbon fiber heating body 130. The present invention selects non-woven fabric 110, sponge 120, and carbon fiber heating body 130 that can be conveniently cut;

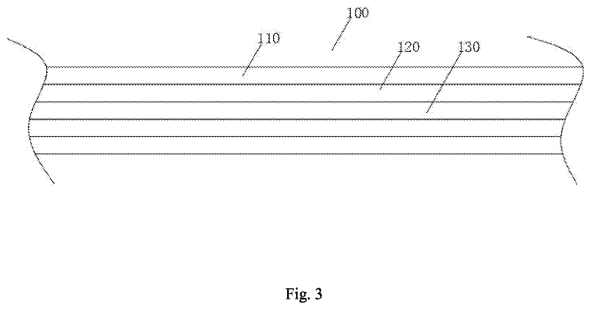

In a specific embodiment, please refer to FIG. 3: the non-woven fabric 110, the sponge 120, and the carbon fiber heating body 130 are cut into the shape of an eye mask. Wherein the non-woven fabric 110 and the sponge 120 have at least two layers; the carbon fiber heating body 130 is placed between the two layers of sponges 120; the two layers of non-woven fabric 110 are sewn outside the two layers of sponges 120 to form an integrated eye mask;It can be quickly cut according to the shape of the eye mask, and then the cut non-woven fabric 110, sponge 120, and carbon fiber heating body 130 can be sewn to form the eye mask main body 100 manufactured by an integral molding process. Moreover, the eye mask host 100 can also be cleaned and maintained during daily use;

Of course, we can also choose different layers of non-woven fabric 110, sponge 120, and carbon fiber heating body 130 to process the eye mask host 100 according to the actual situation, not limited to two layers of non-woven fabric 110, sponge 120, and one layer of carbon fiber heating body 130.

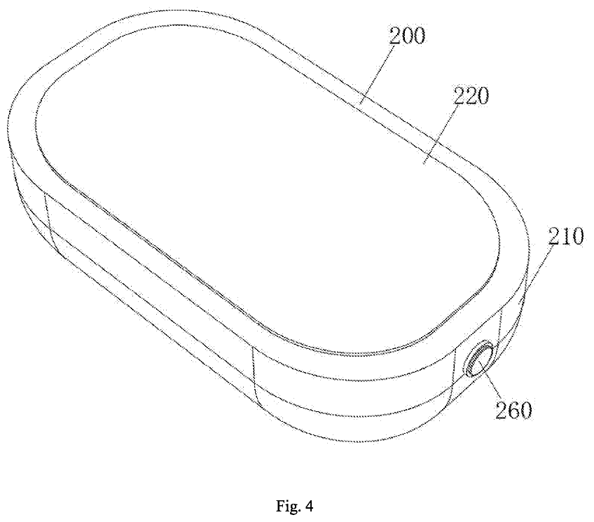

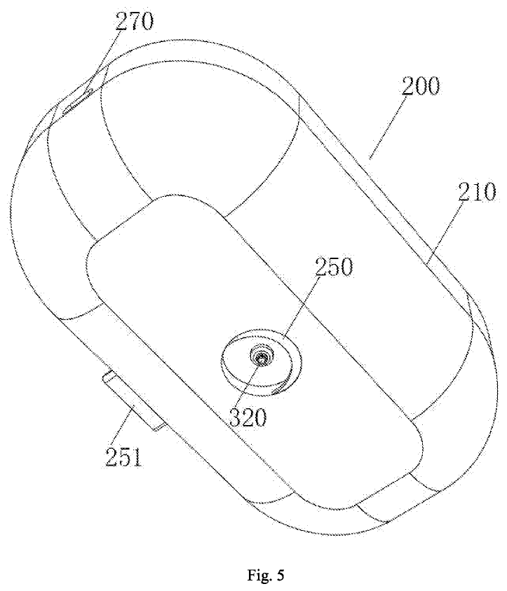

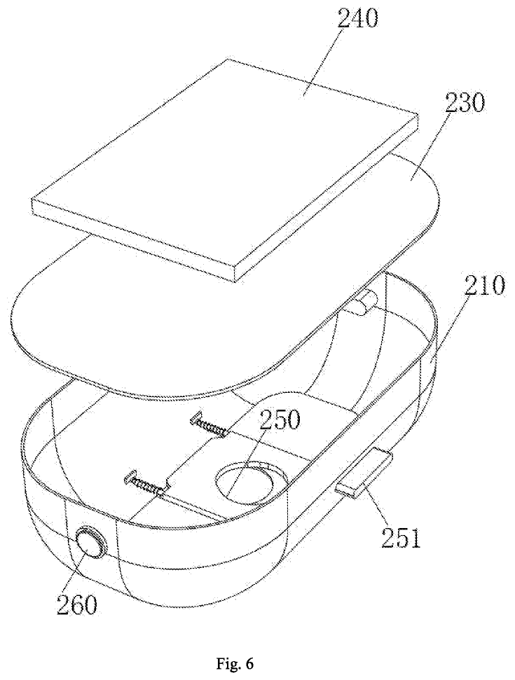

Please refer to FIG. 1, FIG. 4, FIG. 5, and FIG. 6: the power supply controller 200 is composed of an outer casing 210, a display panel 220, a PCB control board 230, a battery 240, and an elastic locking mechanism 250, wherein the elastic locking mechanism 250 is connected to the eye mask The socket 140 provided on the outside of the host 100 is fastened;

In a specific embodiment, the power supply controller 200 is composed of an outer casing 210, a display panel 220, a PCB control board 230, a battery 240, and an elastic locking mechanism 250. This makes the overall volume of the power supply controller 200 smaller and highly integrated. When the present invention is in use, the elastic locking mechanism 250 is used to buckle with the socket 140 on the outside of the eye mask main body 100, so that the power supply controller 200 and the eye mask main body 100 can be disassembled and assembled conveniently.

FIG. 4-7: In this embodiment, the display panel 220 is mounted on the front of casing 210. The PCB control board 230, the battery 240, and the elastic locking mechanism 250 are all installed in the housing 210. A power switch 260 and a charging port 270 are installed on both sides of the battery of the casing 210 respectively, wherein the display panel 220, the power switch 260, and the charging port 270 are all electrically connected to the PCB control board 230. The PCB control board 230 is electrically connected to the battery 240. The display panel 220 can display the electric quantity of the storage battery 240 and the working state of the eye mask, and the PCB control board 230 can be customized as required; this is a mature known technology, and the power switch 260 provided can supply and disconnect the carbon fiber heating body 130 and the working mode are controlled; the charging port 270 provided charges the storage battery 240.

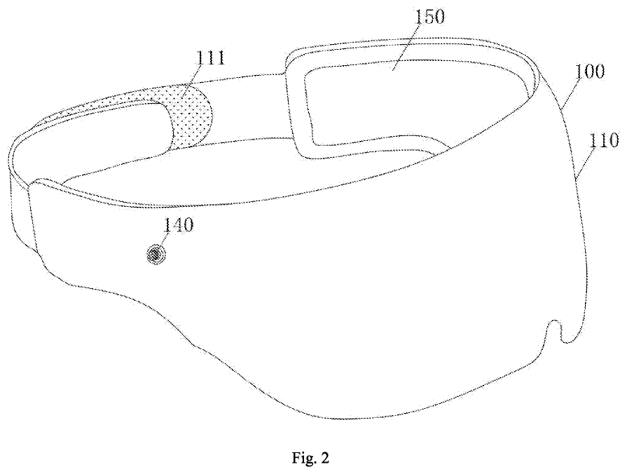

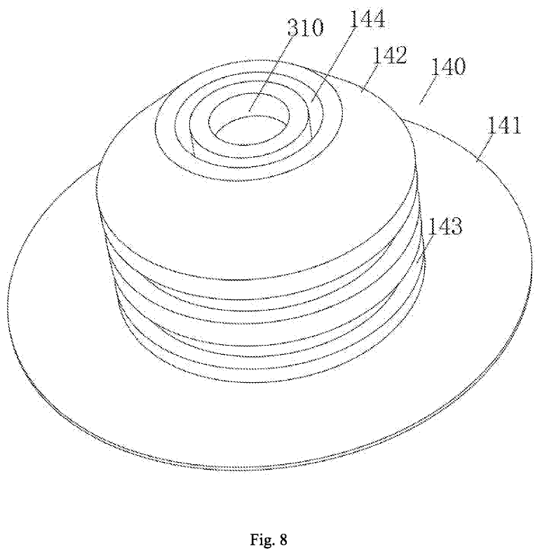

Please refer to FIG. 2, FIG. 5, and FIG. 8: the plug-in power supply mechanism 300 is composed of a female head 310 installed inside the socket 140 and a male head 320 installed inside the outer shell 210; the male head 320 and the female head 310 are plugged Then provide electric energy for the carbon fiber heating body 130 and make the eye mask host 100 generate heat.

In a specific embodiment, the female connector 310 can be a waterproof female connector to prevent the female connector 310 from being damaged during cleaning and maintenance of the eye mask host 100; While supplying power, the elastic locking mechanism 250 is in a buckled state with the socket 140; This can prevent the male connector 320 from being separated from the female connector 310, and also allow the power supply controller 200 to hang on one side of the eye mask host 100.

Please refer to FIG. 8: the socket 140 includes a base 141 fixedly mounted on the outer non-woven fabric 110; the top of the base 141 is fixedly connected with a boss 142; A slot 144 is left; the female head 310 is fixedly installed inside the slot 144.

In a specific embodiment, the base 141 can be fixed on the outer non-woven fabric 110 in a snap-like installation manner; an annular groove 143 is provided on the side of the convex seat 142; this makes the socket 140 and the elastic lock The mechanism 250 can be buckled and fixed, and can also rotate after the elastic locking mechanism 250 is buckled with the socket 140, preventing the socket 140 from being dragged from the non-woven fabric 110 due to the rotation of the power supply controller 200 after being stressed. down to break away.

Please refer to FIG. 4-8: the elastic locking mechanism 250 includes a pressing block 251 plugged into the top of the outer shell 210; one end of the pressing block 251 is connected with a sliding piece 252 placed inside the outer shell 210;the bottom end of the sliding piece 252 is fixedly connected with two guide rods 253; the outside of the guide rods 253 is covered with a return spring 254 fixedly connected to the inside of the outer casing 210;a locking hole 255 is defined in the middle of the sliding piece 252; an edge of the locking hole 255 engages with the annular groove 143.

In a specific embodiment, the sliding piece 252, the guide rod 253, and the locking hole 255 are stamped and processed from a whole metal plate. In this way, the overall structural strength can be effectively guaranteed, and the manufacturing and processing are convenient.By pressing block 251, the sliding piece 252 moves down in the outer shell 210, so that the edge of the locking hole 255 slides out from the annular groove 143, and then the user can conveniently disassemble the power supply controller 200. We release the pressing block 251 again, and the return spring 254 sleeved on the outside of the guide rod 253 can push the slide plate 252 to move upward and reset.

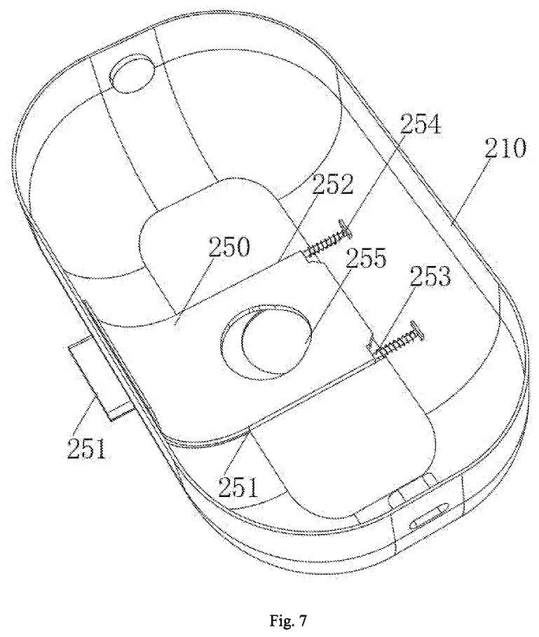

For a specific embodiment, please refer to FIG. 7. There is a guide chute 280 inside housing 210 facing slide 252; the back of slide 252 is slidably connected to the guide chute 280; it can be guided when sliding 252 slides up and down by using the set guide chute 280. Prevent slide sheet 252 from shaking left and right.

The manufacturing process of the eye mask 10 with an integrally formed heat-generating structure is used to manufacture any one of the above eye masks. Specifically include the following steps:

S1, the present invention selects two layers of non-woven fabric 110, two layers of sponge 120 and carbon fiber heating body 130 for cutting, and cuts it into the shape of an eye mask;

S2, the present invention sews the cut non-woven fabric 110, the sponge 120 and the carbon fiber heating body 130 to form an integrated eye mask with a heating function;

S3, the present invention sews Velcro 111 on both ends of the inner non-woven fabric 110, by sewing the Velcro 111, it is convenient for the user to wear the eye mask, and at the same time, the tightness of the eye mask can be adjusted;

S4, the present invention installs the socket 140 with the female head 310 on the outer non-woven fabric 110;

S5, the present invention buckles the elastic locking mechanism 250 of the power supply controller 200 with the socket 140 to complete the detachable installation of the power supply controller 200.

In this embodiment, the female connector 310 is electrically connected to the carbon fiber heating body 130 through wires, so that the carbon fiber heating body 130 can be powered after the female connector 310 is plugged into the male connector 320.

Please refer to FIG. 1-FIG. 2: the inner side of the eye mask main body 100 is reserved with a groove 150 for placing an application bag.

In a specific embodiment, we can use the eye mask host 100 alone; during use, an ice pack can be placed inside the groove 150. When used with ice packs, we apply ice to the eyes, soothe soreness and puffiness around the eyes and better release eye pressure.

The above are only preferred embodiments of the present invention and are not intended to limit the present invention. Various modifications and variations of the present invention will occur to those skilled in the art. Any modifications, equivalent replacements, improvements, etc. made within the spirit and principles of the present invention shall be included within the protection scope of the present invention.

1. The eye mask with the integrally formed heating structure of the present invention is characterized in that it comprises:
An eye mask main body (100) manufactured by an integral molding process, the eye mask main body (100) is sewn from non-woven fabric (110), sponge (120), and carbon fiber heating body (130);
Removable power supply controller (200). The power supply controller (200) is composed of an outer casing (210), a display panel (220), a PCB control board (230), a battery (240), and an elastic locking mechanism (250). Wherein the elastic locking mechanism (250) is fastened to the socket (140) provided outside the eye mask host (100);
Plug-in power supply mechanism (300). The plug-in power supply mechanism (300) is composed of a female head (310) installed inside the socket (140) and a male head (320) installed inside the outer casing (210). After plugging the male connector (320) and the female connector (310), it provides electric energy for the carbon fiber heating body (130) and makes the eye mask host (100) generate heat.

2. The eye mask of the one-piece heat-generating structure according to claim 1. It is characterized in that: the non-woven fabric (110), the sponge (120), and the carbon fiber heating body (130) are cut into the shape of an eye mask, wherein the non-woven fabric (110) and the sponge (120) have at least two layers, and the carbon fiber heating body (130) is placed between two layers of sponges (120), and two layers of non-woven fabrics (110) are sewn on the outside of the two layers of sponges (120) to form an integrated eye mask.

3. The eye mask of the one-piece heat-generating structure according to claim 1. It is characterized in that: the socket (140) includes a base (141) fixedly installed on the outer non-woven fabric (110); the top of the base (141) is fixedly connected with a convex seat (142); the convex seat An annular groove (143) is opened on the edge of (142); a slot (144) is reserved in the middle of the boss (142), wherein the female head (310) is fixedly installed inside the slot (144).

4. The eye mask of the one-piece heat-generating structure according to claim 1. It is characterized in that: the display panel (220) is installed on the front of the outer casing (210), and the PCB control board (230), battery (240), and elastic locking mechanism (250) are all installed inside the outer casing (210); A power switch (260) and a charging port (270) are respectively installed on both sides of the outer casing (210).

5. The eye mask with the integrated heating structure according to claim 1, is characterized in that: the elastic locking mechanism (250) includes a pressing block (251) inserted into the top of the housing (210); one end of the pressing block (251) is connected to The slider placed in the housing (210) is connected. Part (252); the bottom end of the sliding part (252) is fixedly connected with two guide rods (253); the outside of the guide rod (253) is covered with a back-moving spring (254). a locking hole (255) is provided in the middle of the sliding piece (252); the edge of the locking hole (255) is engaged with the annular groove (143).

6. The eye mask with a one-piece heat-generating structure according to claim 1, is characterized in that: there is a guide chute (280) inside the outer shell (210) facing the sliding piece (252); The back side of the sheet (252) is slidably connected with the guide chute (280).

7. A manufacturing process for an eye mask with a body-molded heating structure is characterized in that it is used to manufacture the eye mask described in any of claims 1. Specifically include the following steps:
S1, the present invention selects two layers of non-woven fabric (110), two layers of sponge (120), and a carbon fiber heating body (130) for cutting, and cuts it into the shape of an eye mask;
S2, the present invention sews the cut non-woven fabric (110), sponge (120), and carbon fiber heating body (130) to form an integrated eye mask host (100) with a heating function;
S3, the present invention sews Velcro (111) on both ends of the inner non-woven fabric (110);
S4, the present invention installs a socket (140) with a female head (310) on the outer non-woven fabric (110);
S5, the present invention buckles the elastic locking mechanism (250) of the power supply controller (200) with the socket (140) to complete the detachable installation of the power supply controller (200).

8. The manufacturing process of an eye mask with an integrally formed heating structure according to claim 7, is characterized in that: the female head (310) is electrically connected to the carbon fiber heating body (130) through a wire.

9. The manufacturing process of an eye mask with an integrally formed heating structure according to claim 7, is characterized in that: a groove (150) for placing an application bag is reserved inside the main body of the eye mask (100).
